(19) United States
(12) Patent Application Publication
(10) Pub. No.: US 2022/0331459 A1
(43) Pub. Date: Oct. 20, 2022

(54) Distributing Microparticles

(71) Applicants: Biocompatibles UK Limited, Surrey (GB); Oxford University Innovation Limited, Oxford (GB)

(72) Inventors: Jonathan James VINCE, West Sussex (GB); Eleanor Phoebe Jane STRIDE, Oxford (GB)

(21) Appl. No.: 17/736,435

(22) Filed: May 4, 2022

(30) Foreign Application Priority Data
May 21, 2020 (GB) GB 2106412.6

(51) Int. Cl. A61K 51/12; A61K 41/00

(52) U.S. Cl. CPC A61K 51/1255; A61K 41/0047

(57) ABSTRACT

A method of distributing microparticles is provided, the method comprising: providing a plurality of microparticles at an insertion site in a medium; applying ultrasound to the insertion site that generates gas bubbles by cavitation at cavitation nuclei located at the insertion site and drives movement of the gas bubbles such that the gas bubbles drive movement of the microparticles into a desired spatial distribution in the tumour. The method may be a method of treating a tumour, and the microparticles may comprise a radioisotope for treating the tumour. Microparticles for use in the treatment of a tumour by the method are also disclosed.

The present invention relates to distributing microparticles, and in some aspects to treatment of a solid tumour by distributing microparticles comprising a radioactive isotope.

Microparticles such as microspheres are used in many applications. For example they may be used as a diagnostic tool in medical assays, or to alter the density of a plastic to provide extra buoyancy. Radioactive microparticles, i.e. microparticles comprising at least one radioisotope, can be used for imaging or for manipulation of matter inside a medium. They are used widely in medical imaging and in the diagnosis of various diseases. In many of these uses, positioning microparticles at their intended location can be difficult.

A particular application of radioactive microparticles is in treatment of tumours. Brachytherapy, and its modern evolution selective internal radiation therapy (SIRT) involves the implantation of radioactive nuclei either as bulk solids (e.g. Iodine crystals) or as colloidal suspensions (e.g. Yttrium citrate suspension) in a liquid medium. SIRT was developed to extend and improve the quality of life of patients with non-resectable hepatocellular carcinomas (HCC), for whom external beam radiotherapy (EBRT) is not suitable due to the liver's poor tolerance of radiation.

Current approaches to SIRT use high purity isotopes of known grade to localise radiation using permanent biocompatible microspheres, with calculated emission energies and treatment durations, for specific indications. As with brachytherapy, SIRT allows for precise, controlled radiotherapy to radiosensitive organs and tissues, which otherwise would not tolerate large dose of unfocused, diffuse radiation. Treatment is minimally invasive, via femoral or radial access, enabling delivery through out-patient care and making it an attractive alternative to EBRT. Despite the initial concept of treatment being developed in the 1950's, adoption and practice of SIRT as a palliative technique did not become widespread until the approval of current SIRT products in the 2000's. SIRT remains a non-curative treatment, recommended by several health agencies across the globe.

The therapeutic effect of SIRT is hampered by the terminal distribution of the radioactive microspheres. Due to the limited depth of radiation penetration produced by beta emission from commercial radioembolics, treatment efficacy is directly correlated with the distribution of the microspheres within the tumorous tissue. Treatment of larger areas requires either a larger number of microspheres or a greater distribution of the same number of spheres, with a decreased intensity of radiation.

The distribution of the microspheres is ultimately limited by the placement of the catheter delivering them. In hypoxic solid tumours large areas of the tumour may remain untreated due to the vasculature of the cancer primarily being present on the periphery of the solid lesion, preventing delivery of radioactive microspheres to the centre of the solid mass. Thus the energy of irradiation and its subsequent depth of irradiation are vital to the viability of the procedure. If the depth of penetration of the radiation were to be extended, to enable treatment of a greater proportion of the tumour, it is expected that patient prognosis would also improve.

According to a first aspect of the invention there is provided a method of distributing microparticles, the method comprising: providing a plurality of microparticles at an insertion site in a medium; and applying ultrasound to the insertion site that generates gas bubbles by cavitation at cavitation nuclei located at the insertion site and drives movement of the gas bubbles such that the gas bubbles drive movement of the microparticles into a desired spatial distribution in the medium.

Surprisingly, it has been found that microscale particles can be driven by bubbles, in particular microbubbles, generated by ultrasound-induced cavitation. Ultrasound-induced cavitation of microbubbles has been used to entrain nanoscale particles in a liquid of the medium, but it was unexpected that ultrasound-induced cavitation of microbubbles can also drive movement of much larger (and heavier) microscale particles. The mechanism of entraining used in the nanoscale regime would not be possible in the very different size and mass regime of microscale particles. However, the present inventors have found that gas microbubbles generated by ultrasound-induced cavitation are able to directly impart kinetic energy to microscale particles, and so drive movement of microparticles. As used herein, cavitating refers to the evolution (i.e. growth) and subsequent oscilliation of bubbles, of various sizes, from the cavitation nuclei. The bubbles may or may not subsequently collapse during the application of the ultrasound.

The applied ultrasound performs two functions, namely generating gas bubbles which are suitable for driving the microparticles by cavitation from the cavitation nuclei; and driving movement of the bubbles. The cavitating bubbles in turn drive movement of the microparticles, distributing the microparticles into a desired spatial distribution. In particular, the microbubbles may cause the microparticles to disperse within the medium and/or translate the microparticles within the medium.

In this way, microparticles can be non-invasively spread from their initial location, avoiding the limited distribution of microparticles discussed above, such as where limited capillary size prevents further spread. In particular, where the method is used to distribute microparticles in a tumour, the microspheres may be spread into a tumour even where vasculature is limited towards the centre of the tumour.

In some embodiments, providing the plurality of microparticles at the insertion site may comprise providing the plurality of microparticles at a location where cavitation nuclei are located. For example, the cavitation nuclei may be endogenous to the insertion site. In other embodiments, both microparticles and cavitation nuclei may be provided to the insertion site, either together as a composition or separately. The cavitation nuclei from which microbubbles are generated may comprise both endogenous nuclei already at the insertion site, and exogenous nuclei provided to the insertion site.

In general exogenous cavitation nuclei may be preferable, as high ultrasound energy may be required to generate bubbles from endogenous cavitation nuclei. This may be due to low numbers of endogenous cavitation nuclei, or inefficiency or a high activation energy when generating bubbles from endogenous cavitation nuclei. The high ultrasound energy required for cavitation from endogenous nuclei may damage the surrounding medium.

The cavitation nuclei may comprise at least one of: microbubbles, nanobubbles, nanodroplets and gas stabilising nanoparticles, such as nanocups or nanocones, i.e. nanoscale gas stabilising shells with a void acting as a cavitation nucleus.

In some embodiments, each microparticle may comprise a radioisotope, such as a beta- or gamma-emitting radioisotope. The radioisotope may be one or more of yttrium-90, iodine-125, copper-64, scandium-44, leutitium-176 or holmium-166. It has been found that the ultrasound approach to driving microparticles provides an efficient and effective method for distributing radioactive microparticles in or around a medium.

In some embodiments the microparticles may comprise a therapeutic agent. The therapeutic agent may comprise one or more radioisotopes, as above.

In some embodiments, the medium may be a tissue, such as human tissue. The tissue may be in or may have been removed from a patient. The tissue may be a tumour or part of a tumour.

In particular embodiments, the method may be a method for treating a tumour by distributing microparticles comprising a radioisotope. The tumour may be a tumour of the liver, brain, pancreas, kidney, lung, throat, neck or gut, and in particular may be a glioma, a glioblastoma, or a meningioma.

According to a second aspect of the invention there is provided a method for treating a solid tumour, the method comprising: providing a plurality of microparticles at an insertion site in a tissue of a patient, wherein of the microparticles comprises at least one radioisotope; applying ultrasound to the injection insertion site that generate gas bubbles from cavitation nuclei located at the insertion site, and drives movement of the gas bubbles such that the gas bubbles drive movement of the microparticles into a spatial distribution for providing radiation to treat the tumour. The insertion site may be within the tumour, or outside of the tumour, e.g. adjacent to the tumour. The spatial distribution may be a distribution through the tumour.

Any embodiment of the first aspect of the invention may be combined with the second aspect, in particular embodiments relating to the nature of the ultrasound, cavitation nuclei, and/or microparticles.

According to a third aspect of the invention there is provided a plurality of microparticles, the microparticles comprising a radioisotope, the microparticles being for use in the treatment of a tumour by the method according to any embodiment of the second aspect of the invention or any embodiment of the first aspect wherein the method is a method of treating a solid tumour.

According to a fourth aspect of the invention there is provided a plurality of cavitation nuclei, the cavitation nuclei being for use in the treatment of a tumour by the method according to any embodiment of the second aspect of the invention or any embodiment of the first aspect wherein the method is a method of treating a solid tumour.

According to a fifth aspect of the invention there is provided a composition comprising a plurality of cavitation nuclei and a plurality of microparticles, the microparticles comprising a radioisotope, for use in the treatment of a tumour by the method according to any embodiment of the second aspect of the invention or any embodiment of the first aspect wherein the method is a method of treating a solid tumour.

According to a sixth aspect of the invention there are provided products comprising a plurality of cavitation nuclei and a plurality of microparticles, the microparticles comprising a radioisotope, as a combined preparation for simultaneous, separate, or sequential use in the treatment of a tumour by the method according to any embodiment of the second aspect of the invention or any embodiment of the first aspect wherein the method is a method of treating a solid tumour. Thus the cavitation nuclei and the microparticles may be supplied and inserted into the body separately, but are used together by application ultrasound to treat the tumour.

According to a seventh aspect of the invention there is provided use of a plurality of microparticles, the microparticles comprising a radioisotope, in the manufacture of a medicament for the treatment of a tumour by the method according to any embodiment of the second aspect of the invention or any embodiment of the first aspect wherein the method is a method of treating a tumour.

According to an eighth aspect of the invention there is provided use of a plurality of cavitation nuclei in the manufacture of a medicament for the treatment of a tumour by the method according to any embodiment of the second aspect of the invention or any embodiment of the first aspect wherein the method is a method of treating a tumour.

According to a ninth aspect of the invention there is provided use of a composition comprising a plurality of cavitation nuclei and a plurality of microparticles, the microparticles comprising a radioisotope, in the manufacture of a medicament for the treatment of a tumour by the method according to any embodiment of the second aspect of the invention or any embodiment of the first aspect wherein the method is a method of treating a solid tumour.

To allow better understanding, embodiments of the present invention will now be described by way of non-limitative example with reference to the accompanying drawings, in which:

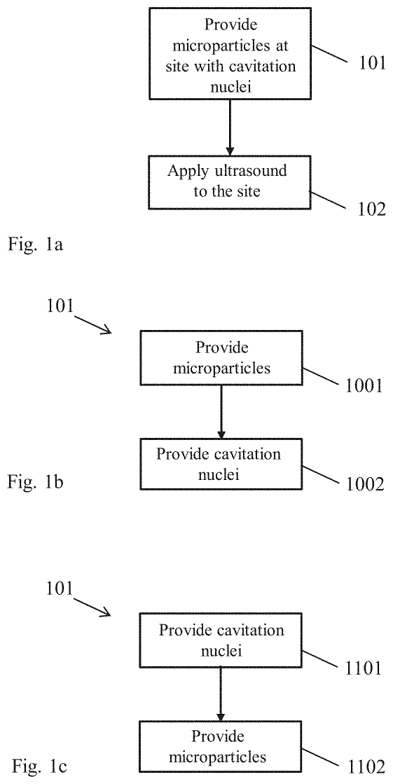

FIG. 1(a) illustrates a method of distributing microparticles according to the present invention;

FIG. 1(b) illustrates a particular example of the first step of the method of FIG. 1(a);

FIG. 1(c) illustrates a further example of the first step of the method of FIG. 1(a)

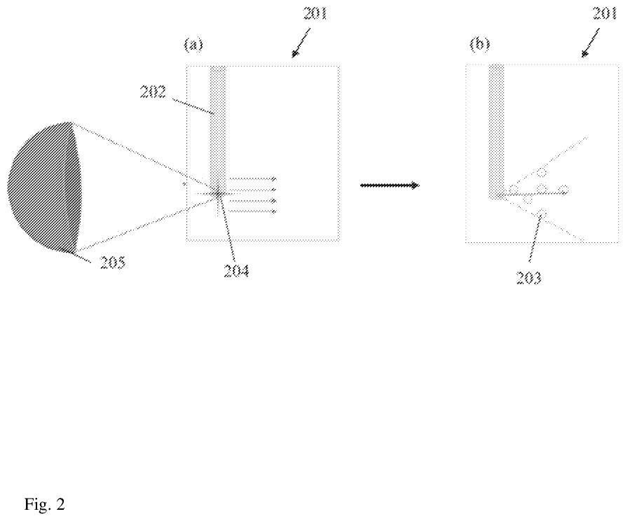

FIG. 2 schematically represents the method of FIG. 1(a) being applied to microparticles;

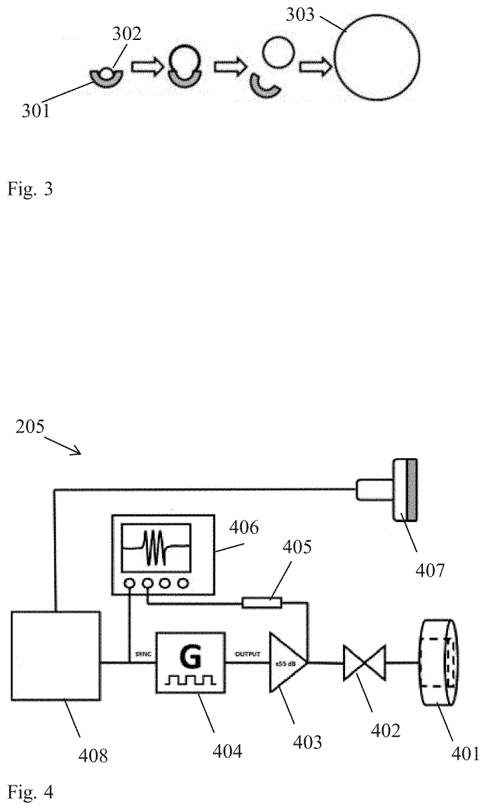

FIG. 3 illustrates bubble formation using nanocups;

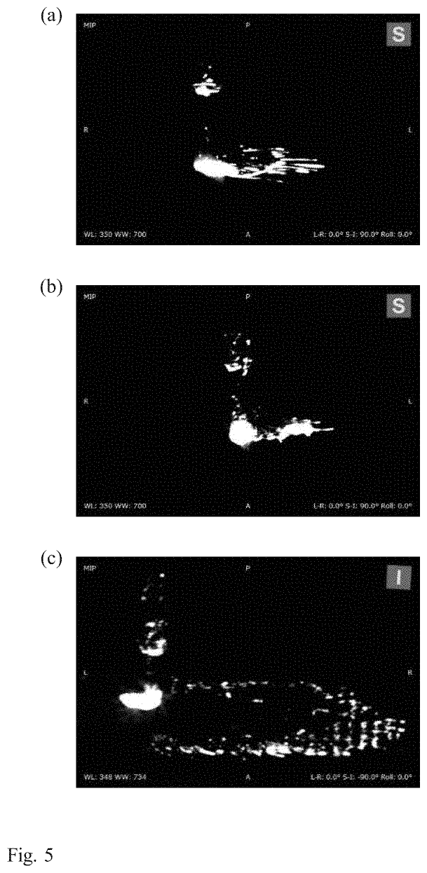

FIG. 4 is a schematic representation of the electronic setup for ultrasound production from a HIFU transducer;

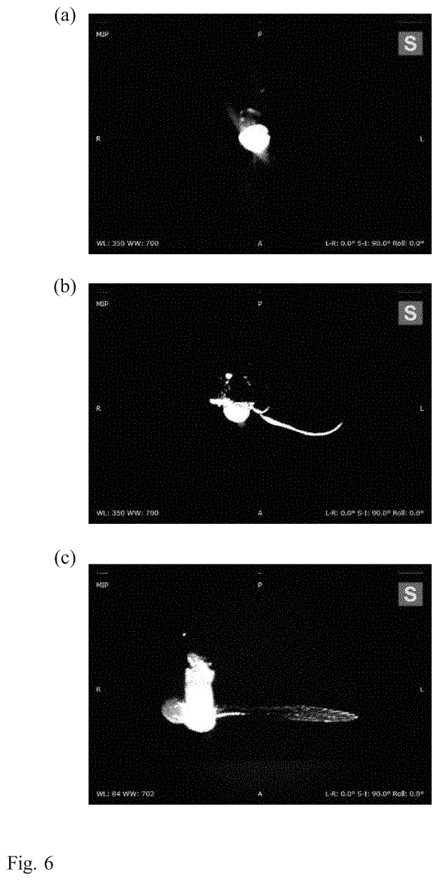

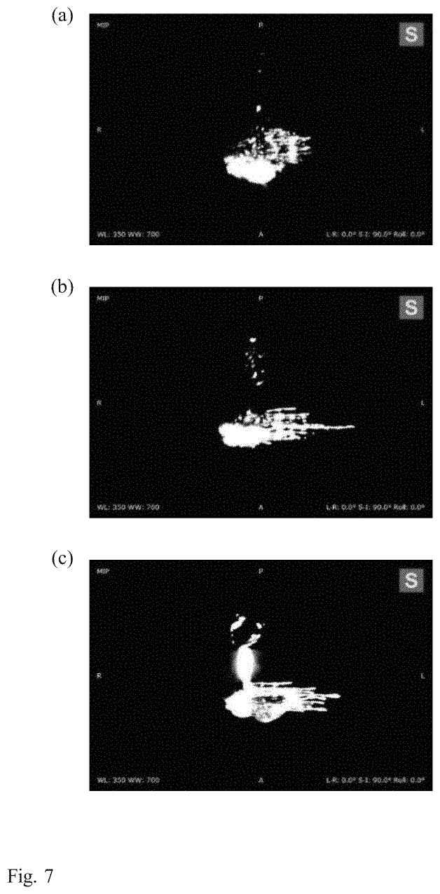

FIGS. 5 and 6 shows μCT images to illustrate the effect of focal pressure on microparticle distribution;

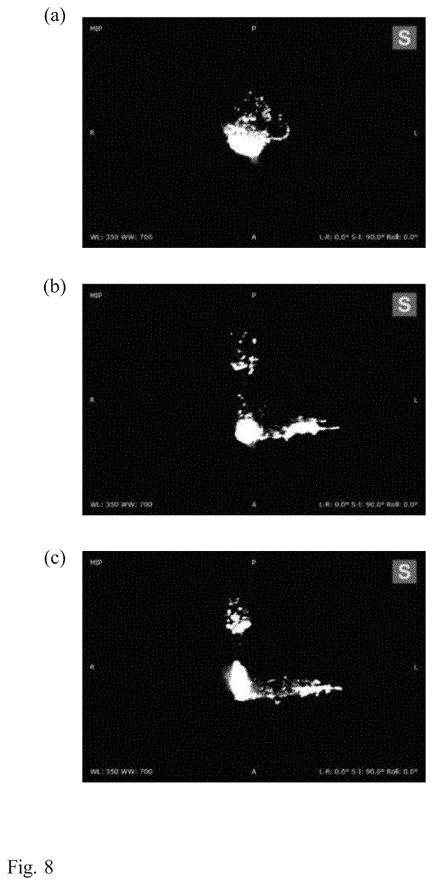

FIG. 7 shows μCT images to illustrate the effect of well diameter on microparticle distribution; and

FIG. 8 shows μCT images to illustrate the effect of duty cycle on microparticle distribution.

FIG. 1(a) illustrates a method of distributing microparticles using ultrasound according to the present invention. As discussed above, it can be difficult to move microparticles to their intended position in a medium. Microparticles may cluster at an insertion site rather than move to a desired distribution, particularly where capillaries are used to introduce microparticles to a medium. The method of FIG. 1(a) improves the distribution of microparticles by applying ultrasound energy.

The method of FIG. 1(a) is used to distribute particles in a medium. The medium may be a material, such as a plastic, in which the microparticles are used to alter a material property such as density or buoyancy or for diagnostic purposes, for example to act as externally detectable radioactive tracers.

The medium may be a tissue, such as a human tissue. The tissue may be in vivo or ex vivo. The microparticles may be used in the tissue for diagnostic purposes (e.g. radioactive tracers) or non-diagnostic and non-treatment purposes (such as altering characteristics of the tissue).

The microparticles may alternatively or additionally be used in the tissue for treating a disease. In particular, the method may be used to distribute particles into a spatial distribution for treating a tumour. The tumour may be a solid tumour. In some embodiments the method may be a method of treating a tumour by distributing microparticles, each microparticle comprising a therapeutic agent such as a radioisotope. In other words, the method may be used to provide selective internal radiation therapy (SIRT).

SIRT has been used to treat a variety of tumour types although it is most commonly used to treat liver cancers such as hepatocellular carcinoma (HCC), cholangiocarcinoma and metastases of colorectal cancer (mCRC) in the liver. Other examples of SIRT treatment include pancreatic neuroendocrine tumours (pNETs), lung tumours and CNS tumours such as gliomas. It would be desirable to enable translation of the SIRT technology into other areas of the body, where external beam radiation is still currently employed as part of first-line treatment and to improve the treatment of tumours already accessible to SIRT. For example, glioblastoma is the second most frequently reported brain tumour, after meningioma, and the most common malignant tumour. Glioblastoma accounts for 15.4% of all primary brain tumours and 45.6% of primary malignant brain tumours in the United States. Patients with glioblastoma multiforme (GBM) have mean survival of 1 year with only 5% of individuals living for 5 or more years, with no prevention strategy or standardised second-line treatment available; these distressing figures are reflective of the limited treatments available, with most procedures resulting in reoccurrence and disease progression from 10 to 30 weeks. By providing a non-invasive ultrasound process to distribute radioactive microparticles into a distribution for treating the tumour, the method of FIG. 1(a) may be used in the treatment of glioblastoma, as well as glioma and meningioma. In general, the method may be used to treat solid tumours of the liver, brain, pancreas, kidney, lung, throat, neck or gut.

Although the following description refers most often to use of microparticles for treating a tumour, it is to be appreciated that the methods discussed may be applied equally to non-treatment uses, such as in non-biological materials.

### Injecting Microparticles

The method of FIG. 1(a) begins at step 101, at which a plurality of microparticles are provided at an insertion site in the medium. The insertion site is also provided with, or already comprises, cavitation nuclei, as discussed in more detail below in relation to FIGS. 1(b) and 1(c). Providing the microparticles may comprise injecting or otherwise inserting a plurality of microparticles at a common location. Where the microparticles are introduced via a capillary or cavity, either naturally occurring or fabricated, the insertion site may be separate from the point at which microparticles are initially introduced into the capillary. For example, the insertion site may be at the end of a capillary. Where the method is used to treat a tumour, providing may comprise providing a plurality of microparticles at an insertion site in a tissue of a patient. The tissue may be the tumour itself, or a tumour remnant (following incomplete excision) or tissue adjacent to or surrounding the tumour or remnant.

FIGS. 2(a) and 2(b) show a schematic example of the method of FIG. 1(a). FIG. 2(a) shows a medium 201 in which microparticles will be distributed. A plurality of microparticles 203 are inserted into a cavity 202, where they gather at an insertion site 204. The microparticles 203 are subsequently distributed in the medium 201 by application of ultrasound from an ultrasound transducer 205, as will be discussed in more detail below in relation to step 102 of the method of FIG. 1(a).

In particular examples, the microparticles may be microspheres. A microsphere is a micron scale particle, which may be either solid or hollow, and which is approximately spherical.

The microparticles may comprise or consist of a ceramic. Such ceramics may comprise a radioisotope, such as yttrium-90 and one or more additional elements such as silicon, aluminium, gallium, strontium manganese or titanium. Since the starting materials are typically be in the form of salts such as oxides or carbonates the ceramic will typically also comprise oxygen. In one approach the ceramic may be a yttrium aluminosilicate glass. Ceramic materials may be particularly useful for providing inert, relatively incompressible microparticles. A particular example, as used for SIRT, is TheraSphere®, manufactured by Biocompatibles UK Ltd (part of Boston Scientific Corp.) TheraSphere® microspheres are a combination of three high purity metal oxides, yttrium, aluminium and silicon which are blended and melted together at extreme temperatures to produce a solid (YAS) glass. The glass is shattered, powdered and spheriodised over a naked flame to form the YAS microspheres (see for example U.S. Pat. No. 4,789,501, which is incorporated herein by reference). Further examples of ceramic SIRT microspheres are described in WO16082045 and WO05087274).

The methods disclosed herein may in general be applied to microparticles, which are particles with a size (which may be a diameter) in a range from 1 μm to 1000 μm. The microparticles may have an average size of 200 μm or less, or preferably 150 μm or less, or more preferably 120 μm or less. The microparticles may have a minimum size of 1 μm or more, or preferably 5 μm or more, or more preferably 10 μm or more, or 20 μm or more, or 50 μm or more, or 100 μm or more. For example, the TheraSphere® microspheres have a size range of 15-35 μm, with a mean size in the range 20-30 μm, which is well-suited to both the present method and to placement in human tissue. The microparticle sizes may be determined by scanning electron microscopy (SEM), optical microscopy, and/or laser diffraction particle size analysis.

As discussed above, radioactive microparticles are particularly useful applications of microparticles. To this end, the microparticles may comprise at least one radioisotope. The radioisotope may a beta- or gamma-emitting radioisotope, such as Yttrium-90, Iodine-125, Copper-64, Scandium-44, leutitium-176 or Holmium-166. In particular, for treatment of tumours, isotope selection is screened against energy emission (electron volts, eV) and efficacy weighed against the systemic toxicity of the radioactive isotope decay product. Iodine-125 and Yttrium-90 are of particular use, due to the poor biological availability of Iodine-125 over Iodine-127 and the inert decay product of Yttrium-90, Zirconium-90. There is a growing trend in the use of atypical beta-emitting isotopes (e.g. Cu-64, Sc-44, Lu-176, Ho-166) for radiotherapy due to their coinciding imaging potential, enabling the monitoring of a procedure in real-time via single photon emission computed tomography (SPECT) and magnetic resonance imaging (MRI).

To produce radioactive microspheres from YAS glass, YAS microspheres comprising Y⁸⁹ are subjected to neutron bombardment in a nuclear reactor, producing Yttrium-90 as the sole radioactive component. Other isotopes formed as the result of the neutron bombardment of silicon or aluminium are deemed stable or insignificant by composition. The enriched Y⁹⁰ isotope undergoes β⁻ decay to Zr⁹⁰ with a half-life of 64.1 h and a decay energy of 0.93 MeV mean energy. Emissions of electrons from the unstable Y⁹⁰ atoms are decelerated by neighbouring atoms electrostatic repulsions; this deceleration and loss of kinetic energy is emitted as ‘braking’ gamma radiation to the surrounding cells (Bremsstrahlung or Cerenkov radiation) and can be detected externally for imaging. The energy emitted into neighbouring cells causes DNA double strand breaks, with downstream signalling of this DNA damage inducing cellular necrosis.

The microparticles may be selected to have a radioactive activity level suitable for providing a clinically acceptable absorbed dose of radiation for the intended treatment. TheraSphere® has an activity per microsphere at calibration of 2500 Bq. In general, the microparticles may have an activity of at least 10 Bq, preferably at least 40 Bq. In some embodiments the activity is at least 500, preferably at least 100 and most preferably at least 2000 Bq per sphere. Maximal activity per sphere is determined by factors such as the selected isotope, number of spheres to be delivered etc. In some embodiments the maximum may be 3000 Bq preferably 5000 Bq, to provide optimum treatment whist minimising damage to surrounding healthy tissue. Total activity of a dose of microspheres may be in the range 3 GBq to 20 GBq.

The target for tumour absorbed dose depends on the susceptibility of the tumour tissue and the surrounding healthy tissue to radiation. An absorbed dose to tumour tissue of at least 50 Gy is desirable, but preferably at least 150 Gy. By way of example, the doses used for liver tumours may be between 50 and 500 Gy, typically at least 150 Gy and preferably at least 200 Gy. The LEGACY study has recently suggested that doses of at least 400 Gy provide a high level of response in liver tumours such as HCC (Ann Oncol. 2020; 31(suppl 4):S692-S693). In the treatment of CNS tumours such as glioma a dose of between 35 Gy and 115 Gy has been reported (Pasciak et al EJNMMI Res. 2020; 10: 96.) and in lung tumours approximately 250 Gy has been used (U.S. Pat. No. 10,232,063). The doses here refer to dose absorbed by the tumour tissue. Dosage can also be measured as dose to perfused tissue—i.e. including the tumour tissue and some surrounding tissue. Dosage to perfused tissue will generally be lower than dosage to tumour tissue.

The microparticles may have a density of 10 g/ml or less, or preferably 5 g/ml or less, or more preferably 4 g/ml or less. The microparticles may have a density of 1 g/ml or more, or preferably 2 g/ml or more, or more preferably 3 g/ml or more. For example, TheraSphere®' microparticles have a relative density of 3.6 gml⁻¹. These comparatively high densities result from using substantially incompressible materials, which is desirable for SIRT and other application of microspheres. The combination of high density and micron size particles results in relatively heavy particles, at least as compared to the nanoparticle regime. Here, density is the density of an individual microparticle. Density may be determined from the crude material of which the microparticles are formed (e.g. glass) prior to spheroidising.

Where the microparticles are used to treat a tumour, the insertion site may be within the tumour. Alternatively, the insertion site may be adjacent to the tumour, for example at a distance sufficiently close to the tumour that the ultrasound energy discussed in step 102 below is able to drive movement of microparticles into the tumour. The insertion site may be on the surface of the tumour, or within 5 cm, or within 2 cm, or with 1 cm of the surface of the tumour. In particular, the insertion site may be within a cavity or capillary formed by excising a portion of the tumour. The method of FIG. 1(a) may comprise excising a portion of the tumour to form the cavity. Alternatively, the insertion site may be at a tumour margin formed by excising a portion of the tumour. The method of FIG. 1(a) may comprise excising a portion of the tumour to form the tumour margin or tumour remnant

Delivery of the microparticles may be performed with sterile 0.9% saline, flushing the spheres out of their v-glass vial residing within a secondary acrylic holder, into the arterial vasculature via microcatheter. The placement of the tip and the internal diameter of the microcatheter is determined by the operator, availability and territory preferences. Delivery may be carried out by catheter to a point in the vasculature that serves the general volume of tissue, rather than the tumour itself. This process relies on the tumour co-opting the local blood supply so that in fact the majority of the microparticles ends up in the tumour, although some percentage still go to the surrounding tissue. Alternatively microparticles may be delivered directly to the blood vessel feeding the tumour. This may be referred to as super selective delivery, as almost all of the microparticles go into the tumour and only limited amounts go to surrounding tissues.

Completion of the irradiation treatment occurs within 2 weeks, with patient follow-up occurring after 6 weeks; consisting of further imaging of the parenchyma and necrotic tissue, potentially with further histology to assess procedural performance.

Cavitation Nuclei

Returning to step 101 of the method, the microparticles 203 are provided at the insertion site 204 where cavitation nuclei are located. A cavitation nucleus can be considered as any material that under exposure to ultrasound, produces an expanding bubble of gas that undergoes either inertial (collapse) or non-inertial cavitation. The expanded bubbles may have at diameter, at maximal extent, in the range 1 μm to 500 μm. As such, the generated bubbles may be termed microbubbles. The size of a bubble may oscillate in accordance with the frequency of the applied ultrasound field. The density of the expanded bubbles will generally be much lower than the density of the microspheres. The density (or mean density) of the expanded bubbles may be in the range 0.5 kg/m³ to 2 kg/m³, or in the range 0.9 kg/m³ to 1.1 kg/m³.

In some embodiments, exogenous cavitation nuclei are provided at the insertion site 204. In some embodiments, step 101 comprises providing a composition of both microparticles 203 and cavitation nuclei to the insertion site 204. FIGS. 1(b) and 1(c) illustrate two alternative embodiments to using a combined composition, in which the cavitation nuclei and microparticles 203 are provided in separate steps.

In the first alternative, shown in FIG. 1(b), step 101 comprises a first step 1001 of providing microparticles 203 at the insertion site 204, for example using the insertion methods discussed above. After inserting the microparticles 204, the method proceeds to step 1002, at which cavitation nuclei are provided to the insertion site 204. The process of providing the cavitation nuclei may be substantially similar to the processes of inserting microparticles discussed above.

In the second alternative, shown in FIG. 1(c), these steps are reversed. Thus in FIG. 1(c), step 101 of the method comprises a first step 1101 of providing cavitation nuclei at the insertion site 204. Subsequently, at step 1102, microparticles 203 are provided at the insertion site 204.

Alternatively, the insertion site 204 to which the plurality of microparticles are provided in step 101 may be a location where cavitation nuclei are already located. In particular, the cavitation nuclei may be endogenous to the insertion site. In such embodiments, there may be no step of providing exogenous cavitation nuclei. Alternatively, exogenous cavitation nuclei may be provided to a site that already has endogenous cavitation nuclei, to enhance the cavitation effect. In general, exogenous cavitation nuclei are preferred, as they tend to require less ultrasound energy to generate the gas bubble. The risk of damage to surrounding tissue caused by the ultrasound is therefore reduced.

Suitable cavitation nuclei include microbubbles, nanobubbles, gas stabilising nanoparticles (e.g. nanocups), and nanodroplets. Each of these is discussed in more detail below. Such cavitation nuclei have been used in combination with ultrasound for nanoscale applications such as drug delivery. These nanoscale particles are on a very different scale to the microparticles of the present invention, particularly the dense, incompressible microspheres used in SIRT. The present inventors have found that, surprisingly, these cavitation nuclei and the bubbles they generate can be applied to the much larger and more massive regime of microparticles, and so help overcome the limited distribution of microparticles in SIRT and other microparticle applications.

### Microbubbles and Nanobubbles

The cavitation nuclei may be or comprise microbubbles (MB) and/or nanobubbles. Microbubbles and nanobubbles are very small pockets of gas, usually containing a perfluorocarbon gas core coated with phospholipids. Under exposure to ultrasound, these gas pockets expand, increasing the diameter bubble. Where the cavitation nuclei comprise microbubbles and/or nanobubbles, generating bubbles by cavitation at the cavitation nuclei comprises growing/evolving the micro/nanobubbles into larger bubbles suitable for driving the microparticles.

Microbubbles have been confirmed occurring naturally in vivo, in swine kidneys and porcine liver and observance of microbubbles are linearly proportional to the concentration of human red blood cells (RBC) in vitro.

Generation of sufficient quantities to produce a cloud of microbubbles in vivo is possible using high intensity focused ultrasound to vaporise liquid at the focal region of the ultrasound transducer (via thermal or mechanical stress).

When exposed to an external ultrasound field, gas filled MB's will expand and contract with an amplitude dependent upon the energy and frequency of the field. At low amplitudes the oscillations of bubbles are largely linear but as the amplitude increases the behaviour of the bubble is increasingly non-linear whereby the radial expansion and contraction may vary significantly with the maximum volume of the MB dependent upon the wave pressure. When the bubble resonance frequency exceeds the ultrasound frequency, the bubbles collapse with the loss of the periodic oscillation denoted as inertial, unstable or transient cavitation. This unstable cavitation event can produce new smaller MBs (nuclei) which have different critical excitation pressures and oscillation frequencies. Under ultrasound exposure a bubble will increase in free energy within the system causing the bubble to increase in temperature and volume, as well as any dissolved gases from the surrounding liquid to coalesce with the bubble to further increase its volume expansion. In theory the expansion of the bubble increases the relative concentration of any entrapped gases in solution at the bubble: liquid interface, defined as rectified diffusion, to further facilitate the influx of gas and the subsequent expansion of MBs.

Synthetic microbubbles primarily consist of a gas core, often perfluorocarbon, stabilised by a lipid or protein shell to prevent the dissolution of gas from larger bubbles (>1 μm) into the surrounding solution; with stabilised bubbles less than 1 μm termed ‘nanobubbles’. With the of core-shell chemistry influencing the stability and cavitation threshold of the microbubble population, often with substrates of interest (cytotoxins, metal particles, proteins, viruses) bound to the surface.

### Gas Stabilising Nanoparticles (Nanocups)

In some embodiments, the cavitation nuclei may be or comprise gas stabilising nanoparticles, in the form of cups or cones. A nanocup is a nanoscale gas stabilising polymer-based cavitation nucleus.

Nanocups can be considered an improvement over microbubbles alone. One of the largest drawbacks of microbubbles is their inability to cross membranes due to their relative size (>1 μm) compared to that of cellular endothelial pores (100-800 nm), with eukaryotic cells typically being in the range of 5-10 μm. With rapid depletion under ultrasound, cavitation events with synthetic microbubbles typically cannot be sustained for more than one to two minutes. The use of nanocups however, can result in a fourfold increase in the duration of sustained cavitation.

Nanometer sized (<1 μm) hollow polymer spheres can be prepared via seeded thermally initiated emulsion polymerisation. As the organic monomers react together, a polymer shell or lens is produced on the surface of the suspended droplets. Due to the osmotic pressure on the unsupported polymer film, the surface shell collapses inwards, producing a polymer disc or ‘nanocup’. Changing the monomer composition changes the viscosity within the particle during polymerisation and the subsequent size of the cavity produced within the nanocup.

FIG. 3 schematically illustrates the process of bubble formation from a nanocup 301. At the first step, the small cavity of the concave polymer nanocup 301 has a nanobubble entrapped on its surface 302 the nucleation sites for cavitation events. In the second step, the gas nanobubble 302 expands radially and outside of the cavity in which it is situated under exposure to ultrasound. In the third step, nanobubble 302 reaches a critical size, where the contact angle between the bubble and the nanocup 301 approaches its maxima, and the nanobubble 302 will dissociate itself from the nanocup 301 (at this point the free nanobubble 302 is likely micron sized and can be thought of as a microbubble). The nanobubble/microbubble 302 continues to expand. Eventually the full bubble 303 is formed. The maximum radius the bubble 303 is independent of the nanocup cavity or acoustic pressure but is directly related to frequency of the focused ultrasound wave. The ultrasound amplitude governs the number of bubbles nucleated and consequently the frequency of non-spherical collapse. Further details of bubble formation from nanocups may be found in Kwan, J. J. et al., “Ultrahigh-Speed Dynamics of Micrometer-Scale Inertial Cavitation from Nanoparticles.” Phys. Rev. Appl. 6, 1-8 (2016), which is incorporated herein by reference.

### Nanodroplets

Unlike microbubbles which have a stabilised gas core, nanodroplets have a liquid (e.g. Perfluoropentane, perfluorohexane) perfluorocabon liquid core which is stabilised by similar lipid or phospholipid molecules on the surface. Nanodroplets offer advantages over microbubbles and nanocups, notably having increased stability, particularly in circulation in vivo, compared to that of their gas counterparts. Upon exposure to ultrasound, the highly volatile liquid is vaporised into a gas, generating a bubble. The energy required for this phase transformation however is far higher energy compared to that of gas core cavitation agents, but still remains a viable option for sensitive therapeutic delivery applications.

### Applying Ultrasound

Returning to the method of FIG. 1(a), the method proceeds to step 102. At step 102, ultrasound is applied to the insertion site 204 to generate bubbles by cavitation at cavitation nuclei located at the insertion site. The applied ultrasound field drives movement of the generated gas bubbles and clouds thereof such that the gas bubbles drive movement of the microparticles into a desired spatial distribution in the medium. The applied ultrasound may cause cavitation of exogenous cavitation nuclei or native cavitation nuclei within the insertion location.

The applied ultrasound performs two functions. It firstly generates bubbles by cavitation at the cavitation nuclei, as discussed above in relation to step 101. It also drives movement of these generated gas bubbles via the acoustic radiation force acting upon them. The cavitating gas bubbles in turn impart kinetic energy to the microparticles, causing the microparticles to move into the desired spatial distribution. The microparticles may disperse and/or translate from their locations at the insertion site. The direction and strength of the ultrasound may be selected to provide a particular movement of the microparticles.

An example of this process is illustrated in FIGS. 2(a) and 2(b). In FIG. 2(a) an ultrasound generator 205 generates a beam of ultrasound focussed on the insertion site 204. This utilises cavitation nuclei at the insertion site 204 (for clarity the bubbles and cavitation nuclei are not illustrated in the figure). As shown in FIG. 2(b), the bubbles drive movement of the microparticles 203, in this case causing them to move into the medium 201 and spread out, yielding a desired spatial distribution of microparticles in the medium 201. The term desired spatial distribution is used herein to refer to a general distribution pattern of the microparticles (e.g. spread out relative to the initial insertion site). The desired spatial distribution depends on the intended use of the microparticles.

It has been found that the combination of ultrasound applied to a site with cavitation nuclei and microparticles is able to distribute microparticle far further and more effectively than conventional methods of inserting microparticles. The maximum displacement of microparticles from the insertion site resulting from this method may be in the range from 1 mm to 4 cm, allowing the microparticles to reach further into tumours than is possible in conventional SIRT methods.

It is noted that the process of driving movement of microparticles with the bubbles is distinct from the mechanism used in nanoscale ultrasound drug delivery. In the latter case moving bubbles cause fluid flow, which in turn entrains nanoparticles. This process of entraining particles is not suitable for the far larger and heavier microparticles considered in the present invention.

Where the method is used for treating a tumour with radioactive microparticles, such as TheraSphere® described above, the microbubbles may drive movement of the microparticles into a spatial distribution for providing radiation to treat the tumour. This may comprise driving the microparticles into the tumour (where the insertion site is in tissue surrounding the tumour) and/or through the tumour. The resulting spatial distribution can penetrate further into the tumour than previously possible, and may be more dispersed within the tumour than would otherwise be possible, greatly increasing the amount of the tumour that can be reached and treated. As a result, a much more effective treatment is possible.

Ultrasound is broadly defined as any frequency above the audible range of humans (20 kHz). The medical applications of conventional ultrasound as a diagnostic imaging modality (sonography) is well established; with most areas of the human body visualised via the varied tissue densities. Medical sonography uses very low energy inputs and frequencies, resulting in a safe and non-destructive methodology suitable for imaging sensitive tissues and organs.

In medical applications, ultrasound waves are most commonly generated using piezoelectric (PZT) ceramics; with the displacement of the elements shape producing an acoustic wave as a series of compressions and decompressions from the transducer surface. In an unfocussed flat transducer the waves will expand as a series of concentric concave wave fronts (a single wavelength apart), undulating in velocity as the wave travels through matter; producing bands of high and low pressure as result. The intensity of sound is relative to the acoustic impedance of the material, viscosity and its elastic behaviour (Young's modulus; elastic or visoelastic). During wave propagation, the particles can either move in the direction of the wave (longitudinal or compressional waves) or orthogonal to the wave (transverse or shear waves).

As the ultrasound wave passes through the tissue or medium, energy is lost either through scattering or heat deposition; with the effective range of the ultrasound constrained by the energy input. The amplitude of the wave slowly dissipates inversely to the propagation distance.

### High Intensity Focussed Ultrasound (HIFU)

In some embodiments, the applied ultrasound is focussed ultrasound, with the focus arranged to be at or near the insertion site. Preferably, the applied ultrasound is high intensity focussed ultrasound (HIFU).

HIFU is achieved by placing a low-velocity confocal lens at the boundary of the PZT element generating the ultrasound, narrowing the wave front to a single controlled focal point at a fixed distance from the flat element, determined by the curvature of the lens. The lens acts by increasing the impedance for the rate of propagation for waves towards the centre of the element, allowing wave fronts (compressions and decompressions) at the edge of the transducer to arrive simultaneously to those at the middle; thus increasing the amplitude of the signal produced at the focal point without increasing the voltage applied to the transducer. Past the focal point (far field), the wave fronts diverge and the signal intensity is lost, with increasing distance.

As a result of focusing the ultrasound wave into a small focal region, a significant pressure gradient is produced compared to that of outside the focal region. The pressure gradient of the acoustic wave acts upon an obstacle within the focus, known as acoustic radiation force (ARF), which can be approximated as:

$$Equation 1 : Acoustic radiation force on an object$$

$$F = \frac{2 AI}{c}$$

F force in (kgs⁻² cm⁻²)
A absorption coefficient of the material (Npcm⁻¹)
I average intensity of the acoustic wave over time at the focus of the ultrasound (Wcm⁻²)
C speed of sound in the medium (cms⁻¹)

Absorption of the energy from the HIFU wave and the conversion into kinetic energy can produce a localised spike in temperature within the tissue, which can result in destruction of tissue. Tissue coagulation, the combination of protein denaturation and permanent cell damage, is induced at 43° C. in vivo. Thermal ablation is correlated to the thermal dose, over 43° C., for a given period of time; with the thermal dose required for ablation varying in tissues and between species. The overall thermotolerance of the brain is particularly low, with each region having its own discrete threshold for damage. Thermal stress is hampered by convection to the surrounding sites; via blood circulation, an increase in cell permeability and perfusion between tissues resulting in a less selective procedure compared to mechanical stress. Pre-focal tissue, despite receiving ultrasound of lower intensity compared to the focal region, does experience pre-focal energy deposition and heating. Pre-focal heating is an important concern for in vivo therapies involving the use of HIFU, where undesired collateral damage can cause thermal ablation prior to the focal point, resulting in skin burns due to the prefocal tissue being continuously exposed to converging HIFU wavefronts.

To provide a balance between effective driving of bubbles and limiting damage to the surrounding tissue, the applied ultrasound in the present method may exert a peak negative focal pressure in the range of 1 to 20 MPa at the insertion site.

### Apparatus

FIG. 4 illustrates an example of an ultrasound setup 205 that may be used to perform step 102 of the method of FIG. 1(a). A transducer 401 may be configured to generate ultrasound with parameters selected by a waveform generator 404 to provide the desired distribution of microparticles. The parameters may be selected to promote the cavitation of bubbles from cavitation nuclei, and desired movement of microbubbles, in order to provide the desired distribution of microparticles. In particular examples, the transducer 401 may generate ultrasound with a fundamental frequency in the range from 0.1 to 5 MHz. The generated ultrasound may have a pulse repetition frequency of in the range from 0.1 to 10 Hz. The generated ultrasound may have a duty cycle of in the range from 1% to 100% (continuous wave). Such parameters have been found to be particularly effective at yielding the desired movement of microparticles, especially in tumour-like media.

The ultrasound generator 205 of FIG. 4 comprises a HIFU transducer 401 with its impedance matching network 402, which is driven by the voltage output of the amplifier 403. In the illustrated example the output voltage of the amplifier 403 is monitored by an oscilloscope 406 using a 1 MΩ high impedance cable 405 to check that there are no anomalies in the transducer drive signal. The input voltage and other ultrasound parameters are controlled by the arbitrary waveform generator 404. Although shown separately in FIG. 4, a linear array 407 is inserted concentrically within the HIFU transducer 401 (represented by the dashed lines within transducer 408). The linear array 407 is used for real time passive acoustic mapping and post exposure imaging, operated by an imaging controller box 408. The imaging controller box 408 is connected to the waveform generator 404 to ensure that the transmit and receive signals are synchronised.

When used for moving microparticles within the body, ultrasound imaging may be used to identify the region to which HIFU pulses should be applied. As the physically co-aligned HIFU transducer 401 and linear array 407 have coincident foci, identifying the imaging focus allows the HIFU target focal point to be identified. Alternatively, an ultrasound probe may be inserted directly into a post-surgical tissue cavity.

### Results

Experiments were performed to investigate distributing TheraSphere microspheres in a hydrogel medium to mimic a tumour. Wells were formed in the medium to act as cavities for delivery of microparticles. The insertion site, at which microparticles were provided, was thus in a well. Ultrasound was applied using the apparatus shown in FIG. 4. Alignment of the transducer and coaxially aligned linear array, proceeded by first aligning the focal depth of the transducer to a centralised 1 mm stainless steel tipped rod, which was inserted into the well to be tested. A pulser receiver was used initially to send a short low energy pulse to the transducer, so that the soft hydrogel was not unnecessarily exposed to HIFU. The transducer was adjusted in 0.1 mm increments until the maximum amplitude of the received signal was observed on the oscilloscope. After which a B-mode image was captured (using a Verasonics® system as the image controller 408) Subsequent wells in the same medium were aligned using the x,y coordinates of the b-mode image, visually confirmed before proceeding with ultrasound exposure. After initial alignment the pulse receiver was replaced with a waveform generator. In this instance the pulse repetition frequency or interval (PRF) was dictated by the Verasonics® instrument and the remaining parameters via the waveform generator.

### Hydrogels as Body Mimicking Tissues

Any tissues whether in vivo, ex vivo or in vitro are unique to the individual organism and are innately variable not only between organisms but within tissues of the same organism. Consequently, producing tissue phantoms which mimic tissue properties over several key characteristics is innately challenging. Hydrogels have been used as in vitro surrogates for mammalian tissue for decades, for ultrasound imaging; with the speed of sound, acoustic attenuation and acoustic impedance analogous to that of serval cancerous indications. Subtle changes can be made to hydrogel compositions to replicate tissue hardness, porosity, acoustic properties as well as cell structure and environment. As a result, hydrogels can simulate a wide variety of properties found in vivo. As hydrogels are largely comprised of water, despite being modelled and tested as solids; their solid content is analogous to many animal tissues, satisfying the requirements for speed of sound (about 1540 m s⁻¹), attenuation (about 0.5 dB cm⁻¹ MHz⁻¹) and backscatter coefficient (in the range from 10⁻⁵ to 10⁻², between 2 and 7 MHz).

Cellular structure is a complex blend of bilayers, microfilaments and tubules, aqueous and organic liquid phases, osmotic pressure and enzymatic catalysis of endogenous matter and it is recognised as near impossible to reproduce in vitro. Hydrogels are therefore a compromise, allowing simulation of the intended investigated material properties akin to their cellular counterpart; hydrogels are not flawless in their design however and replicating one in vivo characteristic, often comes at the expense of another material property due to the complexity of imitating cell structure e.g. replicating the stiffness of a material to simulate a tissue, will frequently compromise the materials permeability. In an attempt to improve the various discrepancies of hydrogels several additives have been assessed including the blending of different polymers (with or without modification) with inorganic additives or inks.

Despite having acoustic properties similar to tissues, hydrogels have a microstructure that varies significantly from that of tissue; having isolated pockets of fluid which redistribute over time under stress within the polymer network Whilst particularly convenient for imitating the material properties of tissues, the fabrication and subsequent consistency of gel models produced are innately variable because of their heterogeneous material properties. Despite the drawbacks of current hydrogel phantoms, gel tissue mimics continue to remain the most suitable surrogate for simulating tissue, due to the efficacy of ex vivo tissue⁷⁵.

For the measurements discussed here an agar based hydrogel was used as the tissue mimic. Agar gels are clear, well documented, less variable and considerably faster to produce compared to PVA freeze-thaw hydrogels. Agar (3 kDa Mw, Sigma Aldrich) was added to deionised water (10 μm, MiliQ®, Type 1) at either 0.5 or 1% w/v, degassed under vacuum for a minimum of 2 hours, before heating the suspension to >85° C. under microwave irradiation before pouring into the phantom mould and allowing to cool at 4° C. for a minimum of 12 hours. Parameter variations and experimental results discussed henceforth are based on 0.5% agar gels.

### Parameters

Nanocups (from Oxsonics®) and Sonovue (Bracco®) were selected as the cavitation agent for the majority of the experiments. SonoVue® was reconstituted and used as instructed by the manufacturer, in the supplied 1-5×10⁸ particlesmL⁻¹ concentration. Nanocups were used in a 1:9 dilution (1.0×10⁹ particlesmL⁻¹), diluting the provided suspension with degassed, filtered deionised water to match the SonoVue® concentration. Both cavitation agents were used in equal volume. 20 mg of YAS glass microspheres (Biocompatible UK Ltd in three size ranges (<15, 15-35, 35+μm) were added to each 2504, injection of diluted nanocups; reducing the chance of sedimentation and phase separation of the cavitation agent from the cold radioembolic.

The pulse repetition frequency was set at 3.3 Hz; this was to allow for equivalent duty cycles over various frequencies investigated, limited by the 50,000 cycle burst of the waveform generator. Each test well was subjected to a total of 3 million cycles, adjusting the ultrasound exposure time and the number of cycles per ultrasound burst as required.

The fundamental frequencies investigated were 0.5, 1.1, 1.5 and 3.3 MHz. Subsequently each frequency has also been tested over several focal pressures; 0.5 MHz (1.4, 3.0, 3.8 MPa), 1.1 MHz (2.6 MPa), 3.3 MHz (7.7, 11.7, 10.9 MPa). Duty cycle (DC) was varied, as with pressure, with each fundamental frequency; 0.5 MHz (2.5%, 5%, 16.5% DC), 1.1 MHz (9.7% DC) and 3.3 MHz (1%, 2.5%, 5% DC).

TheraSphere® glass is colourless and transparent, with individual microspheres difficult to visualise under brightfield microscopy. However due its high relative density (3.4 gcm⁻³) similar to that of human bone, it is possible to visualise the ceramic via x-ray tomography. Under x-ray exposure, objects with higher density appear brighter in the image, according to the Grey scale and corresponding Hounsfield value.

X-ray micro-computer tomography (μCT) images were taken after application of the ultrasound to investigate distribution of the microspheres. μCT images of the wells were generated from 512 sequential image slices (DICOM, 512×512 pixels), which are overlaid in the z-plane, to create a 512×512×512 voxel image, with x-ray acquisition settings saved within the file image stack meta data. Once reconstructed, the distribution of the microspheres was manually measured in a DICOM file viewer, but are currently limited by operator interpretation and bias.

FIG. 5 show μCT images to illustrate the effects of increasing the focal pressure of the applied ultrasound. In FIG. 5(a) the peak negative focal pressure was 1.4 MPa. In FIG. 5(b) the focal pressure was 3.0 MPa. In FIG. 5(c) the peak negative focal pressure was 3.8 MPa. In all cases the fundamental frequency was 0.5 MHz, 3.3 Hz PRF. In FIGS. (a) and (b) the DC was 5%, in FIG. 5(c) the DC was 16.5%. Ultrasound was applied from left to right across all images.

FIG. 6 shows for a fundamental frequency of 3.3 MHz, with peak negative focal pressures 7.7 MPa (6(a)), 11.7 MPa (6(b)) and 10.9 MPa (6(c)). A 5% DC, 3.3 Hz PRF, and 4 mm channels were used. Agar cracking (5(b)0) at 11.7 MPa and planar fission at 10.9 MPa (5(c)) were observed. Pre-focal extravasation was observed at 11.7 MPa (5(b)) and 10.7 MPa peak negative focal pressure (5(c)). Ultrasound applied from left to right across all images.

These figures show that increasing the peak negative pressure at the focal region of the HIFU increases the depth of penetration of microspheres. Reduction in peak negative pressure observed (6(c)) is due to non-linearity of the ultrasound field. Lower frequencies (0.5 MHz) appear to have more substantial effects on a greater proportion of the deposited microspheres, primarily thought to stem from the size of the focal region to which the spheres are exposed. Higher fundamental frequencies produce more discrete, smaller channels of extravasated microspheres rather than some of the omnidirectional bursts seen at lower frequencies; correlating to the size of the transducer focus which decreases with increasing frequency

FIG. 7 shows the influence of well diameter on microsphere distribution. The well diameters were 4 mm (7(a)), 2 mm (7(b)) and 1 mm (7(c)). Ultrasound parameters were: 0.5 MHz 3.3 Hz PRF, 5% DC. 35 μm TheraSphere® microspheres were used. Ultrasound field applied from left to right across all three images.

Whilst not a primary parameter, well diameter does appear to have an influence on the distribution of microsphere projections, particularly with low fundamental frequencies where the focal region encompasses the majority of the well's volume.

FIG. 8 shows similar images to FIG. 7, but with a 2.5% DC rather than 5% to show the effect of DC on microsphere distribution. 15-35 μm TheraSphere® microparticles were used. Below 1% DC no extravasation of microspheres was seen, when compared to control samples not exposed to ultrasound.

Together, these results show that the application of ultrasound to the combination of microspheres and cavitation nuclei does distribute microparticles into the tumour-like medium, indicating that the present invention can improve treatment of tumours by distributing microparticles more effectively than is possible in conventional SIRT methods.

1. A method of distributing microparticles, the method comprising:
providing a plurality of microparticles at an insertion site in a medium; and
applying ultrasound to the insertion site that generates gas bubbles by cavitation at cavitation nuclei located at the insertion site and drives movement of the gas bubbles such that the gas bubbles drive movement of the microparticles into a desired spatial distribution in the medium.

2. The method of claim 1, wherein the microparticles are microspheres.

3. The method of claim 1, wherein the microparticles of the plurality of microparticles have an average size of 200 μm or less and/or wherein the microparticles of the plurality of microparticles have an average size of 1 μm or more.

4. (canceled)

5. The method of claim 1, wherein the microparticles of the plurality of microparticles have a density of 10 g/ml or less and/or wherein the microparticles of the plurality of microparticles have a density of 1 g/ml or more.

6. (canceled)

7. The method of claim 1, wherein the microparticles comprise a ceramic.

8. The method of claim 1, wherein the microparticles comprise at least one radioisotope.

9. The method of claim 8, wherein the radioisotope is a beta- or gamma-emitting radioisotope.

10. The method of claim 9, wherein the radioisotope is yttrium-90, iodine-125, copper-64, scandium-44, leutitium-176, or holmium-166.

11. The method of claim 10, wherein the microparticles comprise a yttrium aluminosilicate glass.

12. The method of claim 8, wherein the microparticles emit radiation with an activity of 10 Bq or more and/or where the microparticles emit radiation with an activity of 5000 Bq or less.

13. (canceled)

14. The method of claim 1, wherein the cavitation nuclei are exogenous to the medium, and wherein the method further comprises providing a plurality of cavitation nuclei at the insertion site.

15. The method of claim 14, wherein a composition comprising the plurality of microparticles and the plurality of cavitation nuclei is provided at the insertion site.

16. The method of claim 14, wherein the plurality of microparticles and the plurality of cavitation nuclei are provided at the insertion site in separate steps.

17. The method of claim 1, wherein the cavitation nuclei are endogenous to the medium.

18. The method of claim 1, wherein the cavitation nuclei comprise at least one of: microbubbles, nanobubbles, nanodroplets and gas stabilising nanoparticles.

19. The method of claim 1, wherein the ultrasound has a fundamental frequency of in the range from 0.1 to 5 MHz, and/or wherein the ultrasound has a pulse repetition frequency of in the range from 0.1 to 10 Hz, and/or wherein the ultrasound has a duty cycle of in the range from 1% to 100%, and/or wherein the ultrasound exerts a peak pressure in the range from 1 to 20 MPa at the insertion site.

20-22. (canceled)

23. The method of claim 1, wherein the medium is a tissue of a patient.

24. The method of claim 23, wherein the method is a method of treating a tumour in the patient, the plurality of microparticles comprise at least one radioisotope, and the spatial distribution is for providing radiation to treat the tumour, each microparticle comprising a radioisotope.

25. The method of claim 24, wherein the insertion site is within the tumour or wherein the insertion site is adjacent to the tumour.

26-28. (canceled)

29. The method of claim 24, wherein the tumour is a solid tumour.

30-40. (canceled)
